(19) United States
(12) Patent Application Publication
(10) Pub. No.: US 2006/0120984 A1
(43) Pub. Date: Jun. 8, 2006

(54) Cosmetic composition comprising at least one silicone copolymer and at least one conditioner, and uses thereof

(75) Inventors: Sandrine Decoster, Saint Gratien (FR); Veronique Douin, Paris (FR); Virginie Bailly, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/336,852

(22) Filed: Jan. 23, 2006

Related U.S. Application Data
(63) Continuation of application No. 09/692,155, filed on Oct. 20, 2000, now abandoned.

(30) Foreign Application Priority Data
Oct. 20, 1999 (FR) 99 13097

(51) Int. Cl. A61K 8/89

(52) U.S. Cl. 424/70.12

(57) ABSTRACT

Cosmetic compositions comprising at least one silicone copolymer with a dynamic viscosity ranging from 1×10⁶ to 100×10⁶ cP and at least one conditioner. This combination can give cosmetic properties, such as at least one of smoothness, lightness, and softness, without the phenomenon of regressing keratin fibers. These compositions can be used for washing and/or conditioning a keratin material, such as the hair or the skin.

The present invention relates to novel cosmetic compositions comprising, in a cosmetically acceptable medium, at least one silicone copolymer defined below, with a dynamic viscosity ranging from 1×10⁶ to 100×10⁶ cP and at least one specific conditioner.

It is well known that hair that has been sensitized (i.e. damaged and/or embrittled) to varying degrees under the action of atmospheric agents or mechanical or chemical treatments, such as dyes, bleaches and/or permanent-waving, can be often difficult to disentangle and to style, and may lack softness.

It has already been recommended to use conditioners, in particular cationic polymers or silicones, in compositions for washing or caring for keratin materials such as the hair, in order to facilitate the disentangling of the hair and to give it softness and suppleness. However, the cosmetic advantages mentioned above can be accompanied, on dried hair, by certain cosmetic effects considered undesirable, i.e., lankness of the hairstyle (lack of lightness of the hair) and lack of smoothness (hair not uniform from the root to the tip).

In addition, the use of cationic polymers for this purpose may have various drawbacks. On account of their high affinity for the hair, some of these polymers can become deposited thereon to a large extent during repeated use, and may lead to adverse effects such as an unpleasant, laden feel, stiffening of the hair and interfiber adhesion which may affect styling. These drawbacks may be more accentuated in the case of fine hair, which lacks liveliness and body.

In summary, it is found that the current cosmetic compositions comprising conditioners are not always entirely satisfactory.

The inventors have now discovered that the combination of at least one silicone copolymer defined below, with a dynamic viscosity ranging from 1×10⁶ to 100×10⁶ cP, with at least one conditioner makes it possible to overcome at least one of these drawbacks.

Thus, after considerable research conducted in this matter, the inventors have found that by introducing at least one silicone copolymer with a dynamic viscosity ranging from 1×10⁶ to 100×10⁶ cP, into compositions, such as hair compositions containing at least one conditioner, it is possible to limit, or even eliminate, at least one of the problems generally associated with the use of such compositions, i.e., for example, the lankness (charged feel following repeated applications) and the lack of smoothness and softness of the hair, while at the same time retaining at least one of the other advantageous cosmetic properties which are associated with conditioner-based compositions.

Moreover, when applied to the skin, for example in the form of a bubble bath or shower gel, the compositions of the invention can provide an improvement in the softness of the skin.

Thus, according to the present invention, cosmetic compositions are now proposed comprising, in a cosmetically acceptable medium, at least one silicone copolymer defined below, wherein said copolymer has a dynamic viscosity ranging from 1×10⁶ to 100×10⁶ cP, and at least one conditioner.

Thus, according to the present invention, novel cosmetic compositions are now proposed comprising, in a cosmetically acceptable medium, at least one silicone copolymer defined below, with a viscosity of between 1×10⁶ and 100×10⁶ cP, and at least one conditioner chosen from:
synthetic oils;
animal oils;
plant oils;
fluoro oils;
perfluoro oils;
natural waxes;
synthetic waxes; and
ceramides of formula (I): 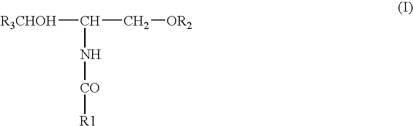
   wherein:
R₁ is chosen from linear and branched, saturated and unsaturated alkyl groups derived from at least one C₁₄-C₃₀ fatty acid, wherein said R₁ is optionally substituted with at least one hydroxyl group in the a position, and wherein said R₁ is optionally substituted with at least one hydroxyl group in the ω position wherein said at least one hydroxyl group is esterified with at least one group chosen from saturated and unsaturated C₁₆-C₃₀ fatty acids;
R₂ is chosen from a hydrogen atom, (glycosyl)ₙ groups, (galactosyl)ₘ groups and sulphogalactosyl groups, wherein n is an integer ranging from 1 to 4 and m is an integer ranging from 1 to 8;
R₃ is chosen from C₁₅-C₂₆ hydrocarbon-based groups, optionally saturated in the 60 position, wherein said R₃ is optionally substituted with at least one C₁-C₁₄ alkyl group;
   with the proviso that when said ceramides of formula (I) are chosen from natural ceramides and natural glycoceramides, R₃ may also be chosen from C₁₅-C₂₆ α-hydroxyalkyl groups wherein the hydroxyl group is optionally esterified with at least one C₁₆-C₃₀ α-hydroxy acid group.

Another subject of the invention relates to the use of at least one silicone copolymer defined below, with a dynamic viscosity ranging from 1×10⁶ to 100×10⁶ cP, in, or for the manufacture of, a cosmetic composition comprising at least one conditioner.

The various subjects of the invention will now be described in detail. All the meanings and definitions of the compounds used in the present invention given below are valid for all the subjects of the invention.

The at least one silicone copolymer results from the addition reaction, in the presence of a catalyst, of at least:
(a) one polysiloxane of formula (I): 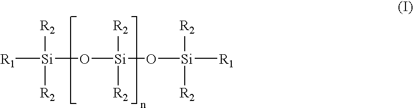
   in which:
R₁, which may be identical or different, are independently chosen from groups that can react by chain addition reaction such as, for example, a hydrogen atom or aliphatic groups comprising an ethylenic unsaturation, such as vinyl, allyl and hexenyl groups;
R₂ in formula (I), which may be identical or different, are independently chosen from hydroxyl, alkyl, alkenyl, cycloalkyl, aryl, and alkylaryl groups, and can optionally further comprise functional groups chosen from ethers, amines, carboxyls, hydroxyls, thiols, esters, sulfonates and sulfates; wherein:
   the alkyl groups comprise, for example, 1 to 20 carbon atoms; the alkenyl groups comprise, for example, from 2 to 10 carbon atoms; the cycloalkyl groups comprise, for example, 5 or 6 carbon atoms; the aryl groups comprise, for example, phenyl groups; and the alkylaryl groups comprise, for example, from 7 to 20 carbon atoms;
   In one embodiment, R₂ is chosen from methyl.
n is an integer wherein the polysiloxane of formula (I) has a kinematic viscosity ranging from 1 to 1×10⁶ mm²/s, for example, n may range from 5 to 5000; and

(b) at least one silicone compound comprising at least one and not more than two groups capable of reacting with the groups R₁ of the polysiloxane (a), wherein:
at least one of the compounds of type (a) and (b) comprises an aliphatic group, such as a C₂-C₆ aliphatic group, comprising an ethylenic unsaturation.

The compounds of type (b) can be another polysiloxane of type (a) in which at least one and not more than two groups R₁ of the polysiloxane (b) can react with the groups R₁ of the polysiloxane (a).

In one embodiment, the at least one silicone copolymer is obtained by addition reaction, in the presence of a hydrosilylation catalyst (for example a platinum catalyst), of at least:
(a) one α,ω-divinylpolydimethylsiloxane, and
(b) one α,ω-dihydrogenopolydimethylsiloxane.

The silicone copolymer generally has a dynamic viscosity, measured at a temperature of about 25° C. and at a shear rate of 0.01 Hz for a stress of 1500 Pa, ranging from 1×10⁶ to 100×10⁶ cP, such as ranging from 5×10⁶ cP to 30×10⁶ cP.

All the dynamic viscosity measurements given in the present patent application were taken at a temperature of about 25° C., on a Carri-Medium CSL2-500 machine.

The kinematic viscosity is measured, for example, at 25° C. according to ASTM standard 445 Appendix C.

The at least one silicone copolymer according to the invention is essentially non-crosslinked, i.e., not crosslinked to an extent sufficient to be referred to as a crosslinked copolymer.

Another subject of the invention relates to compositions wherein the at least one silicone copolymer present in the composition is in the form of an aqueous emulsion.

The expression “aqueous emulsion” means an emulsion of oil-in-water type in which the at least one silicone copolymer is dispersed, such as in the form of particles or droplets, in the aqueous phase forming the continuous phase of the emulsion. This emulsion can be stabilized with a common emulsifying system.

This silicone emulsion can have a silicone droplet or particle size ranging from 10 nm to 50 μm, such as from 0.3 μm to 20 μm. The particle size is measured by laser granulometry.

The emulsifying system comprises at least one surfactant commonly used in silicone emulsions. These surfactants may be nonionic, cationic, anionic or amphoteric, or mixtures thereof, such as those described below.

The emulsifying system represents, for example, from 0.5% to 10% by weight relative to the total weight of the emulsion.

The synthesis of these silicone emulsions is described for example in patent application EP-A-874 017, the disclosure of which is incorporated by reference herein.

Such emulsions are sold for example under the name DC2-1997 Cationic Emulsion by the company Dow Corning. This emulsion comprises an α,ω-divinyl-dimethicone/α,ω-dihydrogenodimethicone copolymer with a dynamic viscosity of about 15×10⁶ cP, an emulsifier of cationic type such as cetyltrimethylammonium chloride, a stabilizer such as hydroxyethylcellulose, and water.

The at least one silicone copolymer can be present in a representative amount ranging from 0.05% to 10% by weight relative to the total weight of the composition, such as from 0.1% to 5% by weight relative to the total weight of the composition.

The aqueous emulsion of the at least one silicone copolymer can be present in a representative amount ranging from 0.5% to 15% by weight relative to the total weight of the composition.

According to the present invention, the at least one conditioner may be in liquid, semi-solid or solid form, such as, for example, oils, waxes or gums.

Suitable synthetic oils may be chosen from polyolefins, such as poly-α-olefins and further such as polyolefins of hydrogenated polybutene type and polyolefins of non-hydrogenated polybutene type. For example, suitable synthetic oils include hydrogenated polyisobutene and non-hydrogenated polyisobutene.

For example, the at least one conditioner may be chosen from isobutylene oligomers having a molecular weight of less than 1000 and mixtures of at least one isobutylene oligomer having a molecular weight of less than 1000 with at least one polyisobutylene having a molecular weight of greater than 1000, such as from 1000 to 15,000.

Non-limiting examples of poly-a-olefins which can be used in the present invention include the products sold under the names Permethyl 99 A, 101 A, 102 A, 104 A (n=16) and 106 A (n=38) by the company Presperse Inc. and the products sold under the name Arlamol HD (n=3) by the company ICI (wherein n denotes the degree of polymerization).

Additional non-limiting examples of suitable synthetic oils include polyolefins of hydrogenated polydecene type and polyolefins of non-hydrogenated polydecene type. Such products are sold, for example, under the names Ethylflo by the company Ethyl Corp. and Arlamol PAO by the company ICI.

The animal and plant oils may be chosen from sunflower oil, corn oil, soybean oil, avocado oil, jojoba oil, marrow oil, grapeseed oil, sesame oil, hazelnut oil, fish oils, glyceryl tricaprocaprylate and plant oils of formula R₉COOR₁₀ and animal oils of formula R₉COOR₁₀ wherein R₉ is chosen from higher fatty acid residues comprising from 7 to 29 carbon atoms and R₁₀ is chosen from linear and branched hydrocarbon-based chains comprising from 3 to 30 carbon atoms, such as, for example, alkyl groups and alkenyl groups. Non-limiting examples of oils of formula R₉COOR₁₀ include purcellin oil and liquid jojoba wax.

Further, according to the present invention, it may be possible to use natural essential oils and synthetic essential oils such as, for example, eucalyptus oil, hybrid lavender oil, lavender oil, vetiver oil, Litsea cubeba oil, lemon oil, sandalwood oil, rosemary oil, camomile oil, savory oil, nutmeg oil, cinnamon oil, hyssop oil, caraway oil, orange oil, geraniol oil, cade oil and bergamot oil.

Suitable waxes may be chosen from natural (i.e. animal and plant) substances that are solid at room temperature (20°-25° C.) and synthetic substances that are solid at room temperature (20°-25° C.). Waxes suitable for use in the present invention may be insoluble in water, soluble in oils and may be capable of forming a water-repellent film.

A definition of waxes may be found, for example, in P. D. Dorgan, Drug and Cosmetic Industry, December 1983, pp. 30-33, the disclosure of which is incorporated herein by reference.

According to the present invention, natural and synthetic waxes comprise carnauba wax, candelilla wax, alfalfa wax, paraffin wax, ozokerite, plant waxes (such as olive tree wax, rice wax, and hydrogenated jojoba wax) and the absolute waxes of flowers (such as the essential wax of blackcurrant flower sold by the company Bertin (France)) and animal waxes (such as beeswaxes and modified beeswaxes (cerabellina)). Other waxes and waxy starting materials which can be used according to the present invention include marine waxes (such as the product sold by the company Sophim under the reference M82), polyethylene waxes and polyolefins in general.

According to the present invention, ceramide-type compounds may be used as the at least one conditioner. The ceramide-type compounds may be chosen from ceramides of formula (I) which comprise natural ceramides of formula (I), synthetic ceramides of formula (I), natural glycoceramides of formula (I), synthetic glycoceramides of formula (I), natural pseudoceramides of formula (I), synthetic pseudoceramides of formula (1), natural neoceramides of formula (I) and synthetic neoceramides of formula (I), and wherein formula (I) has the following structure: 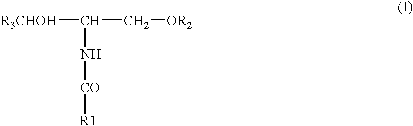
wherein:
R₁ is chosen from linear and branched, saturated and unsaturated alkyl groups derived from at least one C₁₄-C₃₀ fatty acid, wherein said R₁ is optionally substituted with at least one hydroxyl group in the a position, and wherein said R₁ may optionally be substituted with at least one hydroxyl group in the X position wherein said at least one hydroxyl group is esterified with at least one group chosen from saturated and unsaturated C₁₆-C₃₀ fatty acids;
R₂ is chosen from a hydrogen atom, (glycosyl)ₙ groups, (galactosyl)ₘ groups and sulphogalactosyl groups, wherein n is an integer ranging from 1 to 4 and m is an integer ranging from 1 to 8;
R₃ is chosen from C₁₅-C₂₆ hydrocarbon-based groups, optionally saturated in the α position, wherein said R₃ is optionally substituted with at least one C₁-C₁₄ alkyl group;
   with the proviso that when said ceramides of formula (I) are chosen from natural ceramides and natural glycoceramides, R₃ may also be chosen from C₁₅-C₂₆ α-hydroxyalkyl groups wherein the hydroxyl group is optionally esterified with at least one C₁₆-C₃₀ α-hydroxy acid group.

In one embodiment, R₁ is chosen from linear and branched, saturated and unsaturated alkyl groups derived from at least one C₁₆-C₂₂ fatty acid, R₂ is a hydrogen atom and R₃ is chosen from saturated linear C₁₅ hydrocarbon-based groups. Non-limiting examples of such ceramides are:
N-linoleyldihydrosphingosine,
N-oleyidihydrosphingosine,
N-palmitoyidihydrosphingosine,
N-stearyldihydrosphingosine,
N-behenyidihydrosphingosine and mixtures of any of the foregoing.

In another embodiment, R₁ is chosen from saturated and unsaturated alkyl groups derived from at least one C₁₄-C₃₀ fatty acid, R₂ is chosen from (galactosyl)ₙ groups and sulphogalactosyl groups and R₃ is a —CH═CH—(CH₂)₁₂—CH₃ group.

For example, the at least one conditioner may be chosen from the product sold under the trade name Glycocer by the company Waitaki International Biosciences.

In yet another embodiment, ceramide-type compounds of formula (I) are chosen from:
2-N-linoleylaminooctadecane-1,3-diol,
2-N-oleylaminooctadecane-1,3-diol,
2-N-palmitoylaminooctadecane-1,3-diol,
2-N-stearylaminooctadecane-1,3-diol,
2-N-behenylaminooctadecane-1,3-diol,
2-N-[2-hydroxypalmitoyl]aminooctadecane-1,3-diol,
2-N-stearylaminooctadecane-1,3,4-triol
N-stearylphytosphingosine,
2-N-palmitoylaminohexadecane-1,3-diol and mixtures of any of the foregoing.

Non-limiting examples of suitable fluoro oils according to the present invention include the perfluoropolyethers described in patent application EP-A486,135, the disclosure of which is incorporated herein by reference, and the fluorohydrocarbon compounds described in patent application WO 93/11103, the disclosure of which is incorporated herein by reference. Other suitable perfluoropolyethers are sold under the trade names Fomblin by the company Montefluos and Krytox by the company Du Pont.

As used herein, “fluorohydrocarbon compounds” refers to compounds comprising a carbon skeleton wherein at least one hydrogen atom has been replaced with a fluorine atom.

The fluoro oils may also be chosen from fluoroamines (such as perfluorotributylamine), fluorohydrocarbons (such as perfluorodecahydronaphthalene), fluoro esters and fluoro ethers.

Fluorohydrocarbon compounds may also be chosen from fluorine-comprising fatty acid esters, such as the product sold under the name Nofable FO by the company Nippon Oil.

According to the present invention, the at least one conditioner is present in an amount generally ranging from 0.001% to 20% by weight, such as from 0.01% to 10% by weight and further such as from 0.1% to 3% by weight, relative to the total weight of the final composition.

The compositions of the invention can also comprise at least one surfactant chosen from anionic, amphoteric and nonionic surfactants, which is generally present in an amount ranging from approximately 0.1% to 60% by weight relative to the total weight of the composition, such as from 3% to 40% and further such as from 5% to 30%.

The at least one surfactant chosen from anionic, amphoteric and nonionic surfactants, which are suitable for carrying out the present invention are, for example, the following:

(i) Anionic Surfactant(s):

In the context of the present invention, their nature is not of critical importance.

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates. The alkyl and acyl radicals of all of these various compounds can for example comprise from 8 to 24 carbon atoms, and the aryl radicals can for example be chosen from phenyl and benzyl groups.

For example, anionic surfactants can be chosen from fatty acid salts such as the salts of oleic, ricinoleic, palmitic and stearic acids, coconut oil acid and hydrogenated coconut oil acid and acyl lactylates in which the acyl radical comprises from 8 to 20 carbon atoms. At least one weakly anionic surfactant can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups.

As a further example, the anionic surfactant can be at least one salt chosen from alkyl sulfate salts and alkyl ether sulfate salts.

(ii) Nonionic Surfactant(s):

Useful nonionic surfactants include compounds that are well known per se (see for example in this respect “Handbook of Surfactants” by M. R. Porter, published by Blackie & Son (Glasgow and London), 1991, pp. 116-178), the disclosure of which is incorporated by reference herein, and, in the context of the present invention, their nature is not a critical feature. Thus, nonionic surfactants can include polyethoxylated, polypropoxylated and polyglycerolated fatty acids, alkylphenols, α-diols and alcohols having a fatty aliphatic chain comprising, for example, 8 to 18 carbon atoms, it being possible for the number of ethylene oxide and propylene oxide groups to range for example from 2 to 50 and for the number of glycerol groups to range for example from 2 to 30. Mention may also be made of copolymers of ethylene oxide and of propylene oxide, condensates of ethylene oxide and of propylene oxide with fatty alcohols; polyethoxylated fatty amides for example comprising from 2 to 30 mol of ethylene oxide, polyglycerolated fatty amides comprising on average 1 to 5, such as from 1.5 to 4, glycerol groups; polyethoxylated fatty amines for example comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides and N-acylaminopropylmorpholine oxides. It will be noted that the alkylpolyglycosides are nonionic surfactants that can be suitable in the context of the present invention.

(iii) Amphoteric Surfactant(s):

Representative amphoteric surfactants, whose nature is not a critical feature in the context of the present invention, can be chosen from aliphatic secondary and tertiary amine derivatives in which the aliphatic radical is chosen from linear and branched chain radicals comprising 8 to 22 carbon atoms and comprising at least one water-soluble anionic group (chosen for example from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbdtaines and (C₈-C₂₀)alkylamido(C₁-C₆)alkylsulfobetaines.

Representative amine derivatives include the products sold under the name Miranol, as described in U.S. Pat. Nos. 2,528,378 and 2,781,354, the disclosures of which are incorporated by reference herein, and having the structures:
R₂—CONHCH₂CH₂—N⁺(R₃)(R₄)(CH₂COO—) (2)
in which:
R₂ is chosen from alkyl radicals derived from an acid R₂—COOH present in hydrolysed coconut oil, heptyl, nonyl and undecyl radicals,
R₃ is chosen from β-hydroxyethyl groups, and
R₄ is chosen from carboxymethyl groups; and
   R₅—CONHCH₂CH₂—N(B)(C) (3)
   in which:
(B) is —CH₂CH₂OX′, with X′ chosen from a —CH₂CH₂—COOH group and a hydrogen atom,
(C) is —(CH₂)_{z}—Y′, with z=1 or 2, and with Y′ chosen from —COOH and —CH₂—CHOH—SO₃H radicals,
R₅ is chosen from alkyl radicals, such as (a) alkyl radicals of an acid R₅—COOH present in oils chosen from coconut oil and hydrolysed linseed oil, (b) alkyl radicals, such as C₇, C₉, C₁₁ and C₁₃ alkyl radicals, and (c) C₁₇ alkyl radicals and the iso forms, and unsaturated C₁₇ radicals.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name Miranol C2M Concentrate by the company Rhône-Poulenc.

In the compositions in accordance with the invention, at least two surfactants of different types can be used. Representative compositions include compositions comprising (a) more than one anionic surfactant, (b) at least one anionic surfactant and at least one amphoteric surfactant, and (c) at least one anionic surfactant and at least one nonionic surfactant. In one embodiment, the composition can comprise at least one anionic surfactant and at least one amphoteric surfactant.

The at least one anionic surfactant used for example, can be chosen from (C₁₂-C₁₄)alkyl sulfates of sodium, of triethanolamine and of ammonium; (C₁₂-C₁₄)alkyl ether sulfates of sodium, of triethanolamine and of ammonium, oxyethylenated with 2.2 mol of ethylene oxide; sodium cocoyl isethionate; and sodium (C₁₄-C₁₆)-α-olefin sulfonate, and used in combination with an amphoteric surfactant chosen from either:

amphoteric surfactants such as the amine derivatives known as disodium cocoamphodipropionate and sodium cocoamphopropionate, sold for example by the company Rhône-Poulenc under the trade name “Miranol C2M Conc®” as an aqueous solution comprising 38% active material, and under the name Miranol C32; or

amphoteric surfactants of zwitterionic type, such as alkylbetaines, for example the cocobetaine sold under the name “Dehyton AB 30” as an aqueous solution comprising 32% AM by the company Henkel.

In one embodiment of the invention, the compositions can also comprise at least one cationic surfactant.

Representative at least one cationic surfactants can be chosen from salts of optionally polyoxyalkylenated primary, secondary and tertiary fatty amines; quaternary, ammonium salts; imidazoline derivatives; and amine oxides of cationic nature.

The at least one cationic surfactants may, for example, be chosen from:

A) quaternary ammonium salts of formula (IV) below: 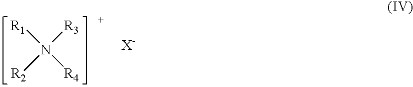
in which:
the radicals R₁, R₂, R₃, and R₄, which may be identical or different, are independently chosen from linear and branched aliphatic radicals comprising from 1 to 30 carbon atoms, and aromatic radicals, such as C₆-C₂₀ aromatic radicals (for example, aryl and alkylaryl), wherein the aliphatic radicals can comprise hetero atoms such as, oxygen, nitrogen, sulfur and halogens, and wherein the aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, comprising from 1 to 30 carbon atoms;
X⁻ is an anion chosen from halides, phosphates, anions derived from organic acids, (C₂-C₆)alkyl sulfates, alkyl sulfonates, and alkylaryl sulfonates.

The compounds of formula (IV) can be chosen from, for example, (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

B) Quaternary ammonium salts of imidazolinium, such as, for example, the salts of formula (V) below: 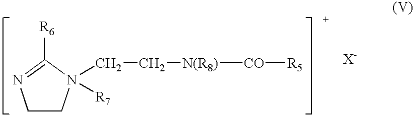
in which:
R₅ is chosen from alkenyl and alkyl radicals comprising from 8 to 30 carbon atoms, for example radicals derived from tallow fatty acid,
R₆ is chosen from a hydrogen atom, C₁-C₄ alkyl radicals, and alkenyl and alkyl radicals comprising from 8 to 30 carbon atoms,
R₇ is chosen from C₁-C₄ alkyl radicals,
R₈ is chosen from a hydrogen atom and C₁-C₄ alkyl radicals,
X⁻ is an anion chosen from halides, phosphates, acetates, lactates, alkyl sulfates, alkyl sulfonates, and alkylaryl sulfonates.
For example, R₅ and R₆, which may be identical or different, are independently chosen from alkenyl and alkyl radicals comprising from 12 to 21 carbon atoms, for example, radicals derived from tallow fatty acid,
R₇ is methyl, and
R₈ is hydrogen.
Such products are, for example, (1) Quaternium-27 (International Cosmetic Ingredient Dictionary and Handbook, hereafter “CTFA”, 1997), i.e., “Rewoquat” W75, W75PG, and W90, and (2) Quaternium-83 (CTFA 1997), i.e., “Rewoquat” W75HPG, which are sold by the company Witco.

C) Diquaternary ammonium salts of formula (VI): 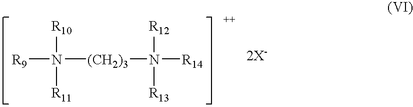
in which:
R₉ is chosen from aliphatic radicals comprising from 16 to 30 carbon atoms,
R₁₀, R₁₁, R₁₂, R₁₃ and R₁₄, which may be identical or different, are independently chosen from a hydrogen atom and alkyl radicals comprising from 1 to 4 carbon atoms, and
X⁻ is an anion chosen from halides, acetates, phosphates, nitrates and methyl sulfates.
For example, such diquaternary ammonium salts can comprise propane tallow diammonium dichloride.

D) Quaternary ammonium salts comprising at least one ester function. The quaternary ammonium salts comprising at least one ester function that can be used according to the invention are, for example, those of formula (VII) below: 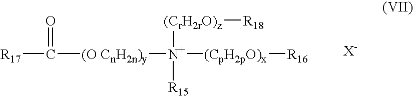
in which:
R₁₅ is chosen from C₁-C₆ alkyl radicals and C₁-C₆ hydroxyalkyl and C₁-C₆ dihydroxyalkyl radicals;
R₁₆ is chosen from:
   acyl groups of the following formula: 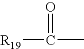
wherein R₁₉ is defined below,
   linear and branched, saturated and unsaturated, C₁-C₂₂ hydrocarbon-based radicals, and
   a hydrogen atom;
R₁₈ is chosen from:
   acyl groups of the following formula: 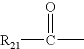
wherein R₂₁ is defined below,
   linear and branched, saturated and unsaturated, C₁-C₆ hydrocarbon-based radicals, and
   a hydrogen atom;
R₁₇, R₁₉, and R₂₁, which may be identical or different, are independently chosen from linear and branched, saturated and unsaturated, C₇-C₂₁ hydrocarbon-based radicals;
n, p and r, which may be identical or different, are independently integers ranging from 2 to 6;
y is an integer ranging from 1 to 10;
x and z, which may be identical or different, are independently integers ranging from 0 to 10;
X⁻ is chosen from simple and complex, organic and inorganic anions;
provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

In one embodiment, the R₁₅ alkyl radicals may be linear and branched and further, for example, linear.

For example, R₁₅ may be chosen from methyl, ethyl, hydroxyethyl and dihydroxypropyl radicals and further for example from methyl and ethyl radicals.

The sum x+y+z may for example range from 1 to 10.

When R₁₆ is chosen from linear and branched, saturated and unsaturated, C₁-C₂₂ hydrocarbon-based radicals, R₁₆ may be long and comprise from 12 to 22 carbon atoms, or short and comprise from 1 to 3 carbon atoms.

When R₁₈ is chosen from linear and branched, saturated and unsaturated, C₁-C₆ hydrocarbon-based radicals, R₁₈ may for example comprise from 1 to 3 carbon atoms.

R₁₇, R₁₉ and R₂₁, which may be identical or different, can, for example, be independently chosen from linear and branched, saturated and unsaturated C₁₁-C₂₁ hydrocarbon-based radicals, and for example from linear and branched, saturated and unsaturated, C₁₁-C₂₁ alkyl and alkenyl radicals.

x and z, which may be identical or different, can for example independently be chosen from 0 or 1.

y for example may be equal to 1.

n, p and r, which may be identical or different, can for example be independently chosen from 2 and 3 and in one embodiment equal to 2.

The anion for example can be chosen from halides (chloride, bromide, and iodide) and alkyl sulfates, such as methyl sulfate. However, methanesulfonate, phosphate, nitrate, tosylate, anions derived from organic acids, such as acetate and lactate, and any other anions compatible with the-ammonium comprising an ester function, may be used.

As a further example, the anion X⁻ can be chosen from chloride and methyl sulfate.

Further examples of ammonium salts of formula (VII) are those in which:
R₁₅ is chosen from methyl and ethyl radicals,
x and y are equal to 1;
z is equal to 0 or 1;
n, p and r are equal to 2;
R₁₆ is chosen from: 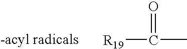
wherein R₁₉ is defined below,
   methyl, ethyl and C₁₄-C₂₂ hydrocarbon-based radicals, and
   a hydrogen atom;
R₁₈ is chosen from: 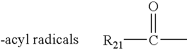
wherein R₂₁ is defined below,
   a hydrogen atom;
R₁₇, R₁₉ and R₂₁, which may be identical or different, are independently chosen from linear and branched, saturated and unsaturated, C₁₃-C₁₇ hydrocarbon-based radicals, such as from linear and branched, saturated and unsaturated C₁₃-C₁₇ alkyl and alkenyl radicals.

The hydrocarbon-based radicals can for example be linear.

Representative compounds of formula (VII) are chosen from diacyloxyethyl-dimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (for example chloride and methyl sulfate). The acyl radicals can for example comprise from 14 to 18 carbon atoms and can for example be obtained from plant oils, such as palm oil and sunflower oil. When the compound comprises several acyl radicals, these radicals, which may be independently chosen, may independently be identical or different.

These products are obtained, for example, by direct esterification of compounds chosen from triethanolamine, triisopropanolamine, alkyldiethanolamines and alkyldiisopropanolamines, which are optionally oxyalkylenated, with fatty acids or with fatty acid mixtures of plant or animal origin, and by transesterification of the methyl esters thereof. This esterification is followed by a quatemization using an alkylating agent such as alkyl halides (such ad methyl and ethyl halides), dialkyl sulfates (for example dimethyl and diethyl sulfates), methyl methanesulfonate, methyl para-toluenesulfonate, glycol chlorohydrin and glycerol chlorohydrin.

Such compounds are sold, for example, under the names Dehyquart by the company Henkel, Stepanquat by the company Stepan, Noxamium by the company Ceca and Rewoquat WE 18 by the company Rewo-Witco.

It is also possible to use the ammonium salts comprising at least one ester function, described in patents U.S. Pat. No. 4,874,554 and U.S. Pat. No. 4,137,180, the disclosures of which are incorporated by reference herein.

Representative quaternary ammonium salts of formula (IV) include tetraalkylammonium chlorides such as, for example, dialkyldimethylammonium chlorides and alkyltrimethylammonium chlorides, in which the alkyl radical comprises from 12 to 22 carbon atoms, for example behenyltrimethylammonium chloride, distearyldimethylammonium chloride, cetyltrimethylammonium chloride, and benzyldimethylstearylammonium chloride, and, stearamidopropyldimethyl(myristyl acetate)ammonium chloride sold under the name “Cepharyl 70” by the company Van Dyk.

According to the invention, the at least one cationic surfactant can for example be present in an amount ranging from 0.1% to 20% by weight relative to the total weight of the final composition, such as from 0.1% to 10%, from 0.5% to 7%, and further such as from 1 % to 5% by weight relative to the total weight of the final composition.

The composition of the invention can also comprise at least one additive chosen from thickeners, fragrances, nacreous agents, preserving agents, silicone sunscreens, non-silicone sunscreens, vitamins, provitamins, cationic, amphoteric, anionic and nonionic polymers, proteins, protein hydrolysates, 18-methyleicosanoic acid, hydroxy, acids, panthenol, volatile and non-volatile, cyclic and linear and crosslinked, modified and non-modified silicones, and any other additive conventionally used in cosmetics which does not substantially adversely affect the properties of the compositions according to the invention.

Generally, these additives are present in the composition according to the invention in amounts, for example, ranging from 0 to 20% by weight relative to the total weight of the composition. The precise amount of each additive is readily determined by those skilled in the art depending on its nature and its function.

The compositions in accordance with the invention can also be used for washing or treating at least one keratin material chosen from hair, skin, eyelashes, eyebrows, nails, lips, scalp, and hair.

The compositions according to the invention can also be a detergent composition chosen from shampoos, shower gels, bubble baths and make-up-removing products. In this embodiment of the invention, the compositions comprise a washing base, which is generally aqueous.

At least one surfactant forms the washing base and can be chosen from anionic, amphoteric, nonionic and cationic surfactants, such as those defined above.

The quantity and quality of the washing base are sufficient to give the final composition at least one of the following qualities, satisfactory foaming power and satisfactory detergent power.

According to the invention, the washing base can be present for example in an amount ranging from 4% to 50% by weight, such as from 6% to 35% by weight and even further such as from 8% to 25% by weight, relative to the total weight of the final composition.

Another subject of the invention is also a process for treating at least one keratin material, such as the skin and the hair, comprising applying a cosmetic composition as defined above to the at least one keratin material and optionally rinsing it out with water.

Thus, this process according to the invention allows maintenance of the hairstyle and treatment of, care of, washing of or removal of make-up from the skin, the hair or any other keratin material.

The compositions of the invention can for example be in forms chosen from rinse-out conditioners and leave-in conditioners; permanent-waving, straightening, dyeing and bleaching compositions; rinse-out compositions to be applied before a procedure chosen from dyeing, bleaching, permanent-waving and straightening the hair; rinse-out compositions to be applied after a procedure chosen from dyeing, bleaching, permanent-waving and straightening the hair; and rinse-out compositions to be applied between the two steps of a procedure chosen from permanent-waving and straightening the hair.

The compositions according to the invention can also be in a form chosen from aqueous and aqueous-alcoholic lotions for a care chosen from skin care and hair care.

The cosmetic compositions according to the invention can be in a form chosen from gels, milks, creams, emulsions, thickened lotions and mousses and can be used for treating at least one keratin material chosen from skin, nails, eyelashes, lips, and hair.

The compositions can be packaged in various forms chosen from vaporizers, pump-dispenser bottles and aerosol containers in order to ensure application of the composition in vaporized form or in the form of a mousse. Such packaging forms are indicated, for example, when it is desired to obtain a spray, a lacquer or a mousse for treating the hair.

In all of the text hereinabove and hereinbelow, the percentages expressed are on a weight basis.

The invention will now be illustrated more fully with the aid of the examples which follow, which should not be considered as limiting it to the embodiments described. In the examples, AM means active material.

### EXAMPLE 1

A conditioner in accordance with the invention, having the following composition, was prepared:

| Mixture of myristyl, cetyl and stearyl | 0.5 | g |
|---|---|---|
| myristate, palmitate and stearate | | |
| Behenyltrimethylammonium chloride | 1.2 | gAM |
| (Genamin KDMP from Clariant) | | |
| N-Oleyldihydrosphingosine | 0.01 | g |
| Cationic emulsion containing 67% | 1.36 | gAM |
| AM of a copolymer of polydimethylsiloxane | | |
| containing α,ω-vinyl groups/ | | |
| polydimethylsiloxane containing α,ω- | | |
| hydrogeno groups (DC-1997 from Dow Corning) | | |
| Mixture of cetyl alcohol and of stearyl | 3 | g |
| alcohol (50/50 by weight) | | |
| Methylalkylalkylamidoethylimidazolinium | 0.05 | gAM |
| methosulphate as a solution containing | | |
| 75% AM in propylene glycol (Rewoquat W | | |
| 75 PG from Rewo) | | |
| Lauryldimethicone copolyol containing | 0.23 | gAM |
| 91% AM (Q2-5200 from Dow Corning) | | |
| Fragrance, preserving agents | qs | |
| Water qs | 100 | g |

This composition is applied to washed and dried hair. It is left to stand on the hair for 2 minutes and is then rinsed off with water.

Hair treated with this conditioner is soft, smooth and disentangles easily.

1-95. (canceled)

96. A cosmetic composition comprising:
(1) at least one conditioner chosen from:
synthetic oils;
animal oils;
plant oils;
fluoro oils;
perfluoro oils;
natural waxes; and
synthetic waxes; and
(2) at least one silicone copolymer with a dynamic viscosity ranging from 1×10⁶ to 100×10⁶ cP, resulting from the addition reaction, in the presence of a catalyst, of:
(a) at least one polysiloxane of formula (II): 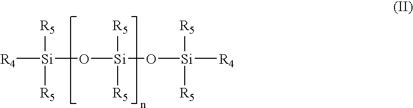
wherein:
R₄, which may be identical or different, are independently chosen from groups that can react by chain addition reaction,
R₅, which may be identical or different, are independently chosen from alkyl, alkenyl, cycloalkyl, aryl, hydroxyl, and alkylaryl groups, and can optionally further comprise at least one functional group,
n is an integer wherein the polysiloxane of formula (II) has a kinematic viscosity ranging from 1 to 1×10⁶ mm²/s; and
(b) at least one silicone compound comprising at least one and not more than two groups capable of reacting with the groups R₄ of the at least one polysiloxane of formula (II), wherein:
at least one of the at least one polysiloxane of formula (II) and the at least one silicone compound (b) comprises an aliphatic group comprising an ethylenic unsaturation;
wherein the composition is in a form chosen from a rinse-out conditioner, a leave-in conditioner, a composition for permanent-waving the hair, a composition for straightening the hair, a composition for dyeing the hair, a composition for bleaching the hair, a rinse-out composition to be applied before a procedure chosen from dyeing, bleaching, permanent-waving and straightening the hair, a rinse-out composition to be applied after a procedure chosen from dyeing, bleaching, permanent-waving and straightening the hair, a rinse-out composition to be applied between the two steps of a permanent-waving operation, and a rinse-out composition to be applied between the two steps of a hair-straightening operation, an aqueous lotion, an aqueous-alcoholic lotion, a gel, a milk, a cream, an emulsion, a thickened lotion, and a mousse.

97. The composition according to claim 96, wherein R₄ is chosen from a hydrogen atom and aliphatic groups comprising an ethylenic unsaturation.

98. The composition according to claim 97, wherein the aliphatic groups comprising an ethylenic unsaturation are chosen from vinyl, allyl and hexenyl groups.

99. The composition according to claim 95, wherein the groups R₅ are chosen from hydroxyl groups; alkyl groups comprising from 1 to 20 carbon atoms; cycloalkyl groups comprising from 5 to 6 carbon atoms; phenyl groups; alkylaryl groups comprising from 7 to 20 carbon atoms; and can optionally further comprise functional groups chosen from ethers, amines, carboxyls, hydroxyls, thiols, esters, sulfonates and sulfates.

100. The composition according to claim 96, wherein in R₅, said alkenyl groups are chosen from alkenyl groups comprising from 2 to 10 carbon atoms.

101. The composition according to claim 96, wherein R₅ is a methyl group.

102. The composition according to claim 96, wherein n is an integer ranging from 5 to 5,000.

103. The composition according to claim 96, wherein the at least one silicone compound of type (b) is chosen from polysiloxanes of formula (II), wherein R₄, R₅, and n are defined as in claim 96 and wherein at least one or two groups R₄ of the at least one silicone compound of type (b) can react with the groups R₄ of the at least one polysiloxane of formula (II) of type (a), with the proviso that that said at least one silicone compound of type (b) differs from said at least one polysiloxane of formula (II) of type (a).

104. The composition according to claim 96, wherein, in the presence of a hydrosilylation catalyst, the at least one silicone copolymer is obtained by addition reaction of at least:
(a) one α,ω-divinylpolydimethylsiloxane, and
(b) one α,ω-dihydrogenopolydimethylsiloxane.

105. The composition according to claim 104, wherein the hydrosilylation catalyst is a platinum catalyst.

106. The composition according to claim 96, wherein said at least one silicone copolymer is in the form of an aqueous emulsion.

107. The composition according to claim 96, wherein the at least one silicone copolymer is essentially non-crosslinked.

108. The composition according to claim 96, wherein the at least one silicone copolymer is present in an amount ranging from 0.05% to 10% by weight relative to the total weight of the composition.

109. The composition according to claim 108, wherein the at least one silicone copolymer is present in an amount ranging from 0.1% to 5% by weight relative to the total weight of the composition.

110. The composition according to claim 106, wherein said aqueous emulsion of the at least one silicone copolymer has a silicone droplet or particle size ranging from 10 nm to 50 μm.

111. The composition according to claim 110, wherein said emulsion of the at least one silicone copolymer has a silicone droplet or particle size ranging from 03 μm to 20 μm.

112. The composition according to claim 96, further comprising at least one cationic surfactant chosen from:
A) quaternary ammonium salts of formula (IV) below: 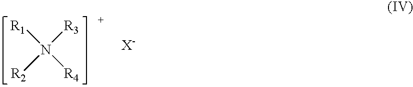
wherein:
R₁, R₂, R₃, and R₄, which may be identical or different, are independently chosen from linear and branched aliphatic radicals comprising from 1 to 30 carbon atoms, and aromatic radicals, wherein the aliphatic radicals optionally comprise hetero atoms, and
X is an anion chosen from halides, phosphates, anions derived from organic acids, (C₂-C₆)alkyl sulfates, alkyl sulfonates, and alkylaryl sulfonates;
B) quaternary ammonium salts of imidazolinium of formula (V) below: 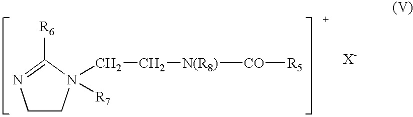
wherein:
R₅ is chosen from alkenyl and alkyl radicals comprising from 8 to 30 carbon atoms,
R₆ is chosen from a hydrogen atom, C₁-C₄ alkyl radicals, and alkenyl and alkyl radicals comprising from 8 to 30 carbon atoms,
R₇ is chosen from C₁-C₄ alkyl radicals,
R₈ is chosen from a hydrogen atom and C₁-C₄ alkyl radicals, and
X⁻ is an anion chosen from halides, phosphates, acetates, lactates, alkyl sulfates, alkyl sulfonates, and alkylaryl sulfonates;
C) diquaternary ammonium salts of formula (VI): 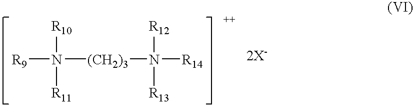
wherein:
R₉ is chosen from aliphatic radicals comprising from 16 to 30 carbon atoms,
R₁₀, R₁₁, R₁₂, R₁₃ and R₁₄, which may be identical or different, are independently chosen from a hydrogen atom and alkyl radicals comprising from 1 to 4 carbon atoms, and
X⁻ is an anion chosen from halides, acetates, phosphates, nitrates and methyl sulfates;
D) quaternary ammonium salts of formula (VII) below comprising at least one ester functional group: 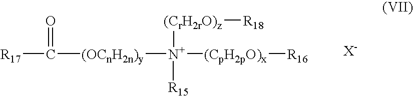
wherein:
R₁₅ is chosen from C₁-C₆ alkyl radicals and C₁-C₆ hydroxyalkyl and C₁-C₆ dihydroxyalkyl radicals;
R₁₆ is chosen from:
acyl groups of the following formula: 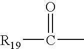
wherein R₁₉ is defined below,
linear and branched, saturated and unsaturated, C₁-C₂₂ hydrocarbon-based radicals, and
a hydrogen atom;
R₁₈ is chosen from:
acyl groups of the following formula: 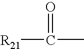
wherein R₂₁ is defined below,
linear and branched, saturated and unsaturated, C₁-C₆ hydrocarbon-based radicals, and
a hydrogen atom;
R₁₇, R₁₉ and R₂₁, which may be identical or different, are independently chosen from linear and branched, saturated and unsaturated, C₇-C₂₁ hydrocarbon-based radicals;
n, p and r, which may be identical or different, are independently integers ranging from 2 to 6;
y is an integer ranging from 1 to 10;
x and z, which may be identical or different, are independently integers ranging from 0 to 10; and
X⁻ is an anion chosen from simple and complex, organic and inorganic anions; and
provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

113. The composition according to claim 112, wherein said at least one cationic surfactant is chosen from:
A) quaternary ammonium salts of formula (IV) below: 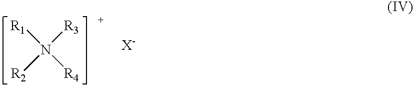
wherein:
X⁻ is an anion chosen from halides, (C₂-C₆)alkyl sulfates, phosphates, alkyl and alkylaryl sulfonates, and anions derived from organic acids, and
i)—the radicals R₁, R₂, and R₃, which may be identical or different, are independently chosen from linear and branched aliphatic radicals comprising from 1 to 4 carbon atoms, optionally comprising hetero atoms, and aromatic radicals, and
R₄ is chosen from linear and branched alkyl radicals comprising from 16 to 30 carbon atoms;
ii)—the radicals R₁ and R₂₁ which may be identical or different, are independently chosen from linear and branched aliphatic radicals comprising from 1 to 4 carbon atoms, optionally comprising hetero atoms, and aromatic radicals, and
R₃ and R₄, which may be identical or different, are independently chosen from linear and branched alkyl radicals comprising from 12 to 30 carbon atoms, wherein said radicals further comprise at least one functional group chosen from ester and amide functional groups.

114. The composition according to claim 112, wherein in said quaternary ammonium salts of formula (VII):
R₁₅ is chosen from methyl and ethyl radicals,
x and y are equal to 1;
z is equal to 0 or 1;
n, p and r are equal to 2;
R₁₀, is chosen from: 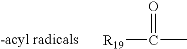
wherein R₁₉ is defined below,
methyl, ethyl and C₁₄-C₂₂ hydrocarbon-based radicals, and
a hydrogen atom;
R₁₈ is chosen from: 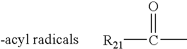
wherein R₂₁ is defined below,
a hydrogen atom; and
R₁₇, R₁₉ and R₂₁, which may be identical or different, are independently chosen from linear and branched, saturated and unsaturated, C₁₃-C₁₇ hydrocarbon-based radicals.

115. The composition according to claim 114, wherein R₁₇, R₁₉ and R₂₁ are chosen from linear and branched, saturated and unsaturated C₁₃-C₁₇ aliphatic radicals.

116. The composition according to claim 114, wherein the hydrocarbon-based radicals are chosen from linear hydrocarbon-based radicals.

117. The composition according to claim 112, wherein the compounds of formula (VII) are chosen from diacyloxyethyidimethylammonium, diacyloxyethylhydroxyethylmethylammonium, monoacyloxyethyidihydroxyethylmethylammonium, triacyloxyethylmethylammonium and monoacyloxyethyihydroxyethyidimethylammonium salts.

118. The composition according to claim 117, wherein said monoacyloxyethyl-hydroxyethyldimethylammonium salts are chosen from monoacyloxyethyl-hydroxyethyldimethylammonium chloride salts and monoacyloxyethyl-hydroxyethyldimethylammonium methyl sulfate salts.

119. The composition according to claim 114, wherein when R₁₆ and R₁₈ are chosen from acyl radicals, said acyl radicals are obtained from plant oils chosen from palm oil and sunflower oil.

120. The composition according to claim 112, wherein X⁻ of said quaternary ammonium salts of formula (IV) is an anion chosen from chloride, bromide, iodide, methyl sulfate, acetate, and lactate.

121. The composition according to claim 112, wherein said aromatic radicals of said quaternary ammonium salts of formula (IV) are chosen from aryl and alkylaryl.

122. The composition according to claim 112, wherein said hetero atoms of said quaternary ammonium salts of formula (IV) are chosen from oxygen, nitrogen, sulfur and halogens.

123. The composition according to claim 113, wherein said aliphatic radicals of formula (IV)(ii) are chosen from alkyl, alkoxy, alkylamide, polyoxy(C₂-C₆)alkylene, and hydroxyalkyl radicals comprising from 1 to 4 carbon atoms.

124. The composition according to claim 113, wherein said R₃ and R₄ of formula (IV)(ii) are chosen from (C₁₂-C₂₂)alkylamido(C₂-C₆)alkyl and (C₁₂-C₂₂)alkylacetate radicals.

125. The composition according to claim 112, wherein said R₅ of formula (V) is chosen from radicals derived from tallow fatty acid.

126. The composition according to claim 112, wherein in said quaternary ammonium salts of imidazolinium of formula (V):
R₅ and R₆, which may be identical or different, are independently chosen from alkenyl and alkyl radicals comprising from 12 to 21 carbon atoms,
R₇ is methyl, and
R₈ is hydrogen.

127. The composition according to claim 126, wherein said R₅ and R₆, which may be identical or different, are independently chosen from radicals derived from tallow fatty acid.

128. The composition according to claim 112, wherein said diquaternary ammonium salts comprise propane tallow diammonium dichloride.

129. The composition according to claim 112, wherein said R₁₅ alkyl radicals of said quaternary ammonium salts of formula (VII) are chosen from linear and branched C₁-C₆ alkyl radicals.

130. The composition according to claim 129, wherein said R₁₅ radicals are linear radicals.

131. The composition according to claim 130, wherein said R₁₅ radicals are chosen from methyl, ethyl, hydroxyethyl and dihydroxypropyl.

132. The composition according to claim 131, wherein said R₁₅ radicals are chosen from methyl and ethyl.

133. The composition according to claim 112, wherein said sum of x+y+z of said quaternary ammonium salts of formula (VIII) ranges from 1 -10.

134. The composition according to claim 112, wherein said quaternary ammonium salts of formula (IV) are chosen from (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

135. The composition according to claim 112, wherein said at least one cationic surfactant is chosen from behenyltrimethylammonium salts, stearamidopropyldimethyl(myristyl acetate)ammonium salts, Quaternium-27 and Quaternium-83.

136. The composition according to claim 112, wherein the at least one cationic surfactant is present in an amount ranging from 0.1 % to 10% by weight relative to the total weight of the composition.

137. The composition according to claim 136, wherein the at least one cationic surfactant is present in an amount ranging from 1 % to 5% by weight relative to the total weight of the composition.

138. The composition according to claim 96, further comprising at least one surfactant chosen from anionic, nonionic, and amphoteric surfactants.

139. The composition according to claim 138, wherein the at least one surfactant chosen from anionic, nonionic, and amphoteric surfactants is present in an amount ranging from 0.1% to 60% by weight, relative to the total weight of the composition.

140. The composition according to claim 139, wherein the at least one surfactant chosen from anionic, nonionic, and amphoteric surfactants is present in an amount ranging from 5% to 30% by weight, relative to the total weight of the composition.

141. The composition according to claim 138, wherein the at least one surfactant chosen from anionic, nonionic, and amphoteric surfactants comprises at least one anionic surfactant salt chosen from alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates.

142. The composition according to claim 138, wherein said at least one surfactant is chosen from anionic surfactants chosen from alkaline salts, sodium salts, ammonium salts, amine salts, amino alcohol salts and magnesium salts.

143. The composition according to claim 141, wherein said alkyl and acyl portions of radicals of said salts comprise 1 and from 8 to 24 carbon atoms, and said aryl portions of radicals of said salts are phenyl.

144. The composition according to claim 138, wherein said at least one surfactant is chosen from anionic surfactants chosen from fatty acid salts, acyl lactates wherein the acyl radical comprises 8 to 20 carbon atoms, and weakly anionic surfactants.

145. The composition according to claim 144, wherein said fatty acid salts are chosen from the salts of oleic acid, ricinoleic acid, palmitic acid, stearic acid, coconut oil acid and hydrogenated coconut oil acid.

146. The composition according to claim 138, wherein said at least one surfactant is chosen from anionic surfactants chosen from alkyl-D-galactosiduronic acids and their salts, polyoxyalkylenated (C₆-C₂₄) alkyl ether carboxylic acids and their salts, polyoxyalkylenated (C₆-C₂₄) alkylaryl ether carboxylic acids and their salts, and polyoxyalkylenated (C₆-C₂₄) alkylamido ether carboxylic acids and their salts.

147. The composition according to claim 144, wherein said weakly anionic surfactants comprise from 2 to 50 ethylene oxide groups.

148. The composition according to claim 141, wherein said at least one anionic surfactant salt is chosen from alkyl sulfates and alkyl ether sulfates.

149. The composition according to claim 138, wherein said at least one surfactant is chosen from nonionic surfactants chosen from polyethoxylated, polypropoxylated and polyglycerolated fatty acids, alkylphenols, a-diols and alcohols having a fatty aliphatic chain comprising from 8 to 18 carbon atoms, wherein the number of ethylene oxide and propylene oxide groups ranges from 2 to 50 and the number of glycerol groups ranges from 2 to 30, copolymers of ethylene oxide and of propylene oxide, condensates of ethylene oxide and of propylene oxide with fatty alcohols, polyethoxylated fatty amides comprising from 2 to 30 mol of ethylene oxide, polyglycerolated fatty amides comprising on average 1 to 5 glycerol groups, polyethoxylated fatty amines comprising from 2 to 30 mol of ethylene oxide, oxyethylenated fatty acid esters of sorbitan comprising from 2 to 30 mol of ethylene oxide, fatty acid esters of sucrose, fatty acid esters of polyethylene glycol, alkylpolyglycosides, N-alkylglucamine derivatives, and amine oxides.

150. The composition according to claim 149, wherein said polyglycerolated fatty amides comprise on average from 1.5 to 4 glycerol groups.

151. The composition according to claim 149, wherein said amine oxides are chosen from(C₁₀-C₁₄)alkylamine oxides and N-acylaminopropylmorpholine oxides.

152. The composition according to claim 149, wherein said nonionic surfactants are chosen from alkylpolyglycosides.

153. The composition according to claim 138, wherein said at least one surfactant is chosen from amphoteric surfactants chosen from aliphatic secondary and tertiary amine derivatives wherein the aliphatic radical is chosen from linear and branched chain radicals comprising from 8 to 22 carbon atoms and comprising at least one water-soluble anionic group, (C₈-C₂₀)alkylbetaines, sulfobetaines, (C₈-C₂₀)alkylamido(C₁-C₆)alkylbetaines, and (C₈-C₂₀)alkylamido(C₁-C₆)alkylsulfobetaines.

154. The composition according to claim 153, wherein said at least one water-soluble anionic group is chosen from carboxylates, sulfonates, sulfates, phosphates and phosphonates.

155. The composition according to claim 153, wherein said amine derivatives are chosen from the compounds:
R₂—CONHCH₂CH₂—N⁺(R₃)(R₄)(CH₂COO⁻) (2)
wherein:
R₂ is chosen from alkyl radicals derived from an acid R₂—COOH present in hydrolysed coconut oil, heptyl, nonyl and undecyl radicals,
R₃ is chosen from β-hydroxyethyl groups, and
R₄ is chosen from carboxymethyl groups; and
R₅—CONHCH₂CH₂—N(B)(C) (3)
wherein:
(B) is —CH₂CH₂OX′, with X′ chosen from a —CH₂CH₂—COOH group and a hydrogen atom,
(C) is —(CH₂)₂—Y′ with z=1 or 2, and with Y′ chosen from —COOH and —CH₂—CHOH—SO₃H radicals,
R₅ is chosen from alkyl radicals and unsaturated C₁₇ radicals.

156. The composition according to claim 155, wherein said alkyl radicals of said R₅ are chosen from (a) alkyl radicals of an acid R₅—COOH present in oils chosen from coconut oil and hydrolysed linseed oil, and (b) C₁₇ alkyl radicals and the iso forms.

157. The composition according to claim 156, wherein said alkyl radicals of said R₅ are chosen from alkyl radicals chosen from C₇, C₉, C₁₁ and C₁₃ alkyl radicals.

158. The composition according to claim 138, wherein said at least one surfactant is chosen from at least two surfactants of different types.

159. The composition according to claim 158, wherein said at least two surfactants of different types are chosen from (a) more than one anionic surfactant, (b) at least one anionic surfactant and at least one amphoteric surfactant, and (c) at least one anionic surfactant and at least one nonionic surfactant.

160. The composition according to claim 138, wherein said at least one surfactant is chosen from anionic surfactants chosen from (C₁₂-C₁₄)alkyl sulfates of sodium, of triethanolamine and of ammonium, (C₁₂-C₁₄)alkyl ether sulfates of sodium, of triethanolamine and of ammonium, oxyethylenated with 2.2 mol of ethylene oxide, sodium cocoyl isethionate, and sodium(C₁₄-C₁₆)-α-olefin sulfonate, and from an amphoteric surfactant chosen from either:
amine derivatives comprising disodium cocoamphodipropionate and sodium cocoa mphopropionate, or
amphoteric surfactants of zwitterionic type.

161. The composition according to claim 160, wherein said amphoteric surfactants of zwitterionic type are chosen from alkylbetaines.

162. The composition according to claim 161, wherein said alkylbetaines are chosen from cocobetaine.

163. The composition according to claim 96, further comprising at least one additive chosen from fragrances, nacreous agents, preserving agents, silicone sunscreens, non-silicone sunscreens, vitamins, provitamins, amphoteric, anionic and nonionic polymers, proteins, protein hydrolysates, 18-methyleicosanoic acid, hydroxy acids, panthenol, volatile and non-volatile, cyclic and linear and crosslinked, and modified and non-modified silicones.

164. The composition according to claim 163, wherein said at least one additive is present in an amount ranging from 0 to 20% by weight relative to the total weight of the composition.

165. The composition according to claim 96, wherein said composition chosen from aqueous and aqueous-alcoholic lotions is suitable for skin care or for hair care.

166. The gel, milk, cream, emulsion, thickened lotion or mousse according to claim 96, wherein said gel, milk, cream, emulsion, thickened lotion or mousse is suitable to be applied to at least one keratin material chosen from skin, nails, eyelashes, lips and hair.

167. The composition according to claim 96, wherein said synthetic oils are chosen from hydrogenated polybutene, non-hydrogenated polybutene, hydrogenated polydecene, and non-hydrogenated polydecene.

168. The composition according to claim 96, wherein said at least one conditioner is chosen from sunflower oil, corn oil, soybean oil, avocado oil, jojoba oil, marrow oil, grapeseed oil, sesame oil, hazelnut oil, fish oils, glyceryl tricaprocaprylate and plant oils of formula R₉COOR₁₀ and animal oils of formula R₉COOR₁₀, wherein R₉ is chosen from higher fatty acid residues comprising from 7 to 29 carbon atoms and R₁₀ is chosen from linear and branched hydrocarbon-based chains comprising from 3 to 30 carbon atoms, natural essential oils and synthetic essential oils.

169. The composition according to claim 168, wherein said R₁₀ is chosen from alkyl groups and alkenyl groups.

170. The composition according to claim 168, wherein said natural essential oils and synthetic essential oils are chosen from eucalyptus oil, hybrid lavender oil, lavender oil, vetiver oil, litsea cubeba oil, lemon oil, sandalwood oil, rosemary oil, chamomile oil, savory oil, nutmeg oil, cinnamon oil, hyssop oil, caraway oil, orange oil, geraniol oil, cade oil, and bergamot oil.

171. The composition according to claim 96, wherein said at least one conditioner is chosen from carnauba wax, candelilla wax, alfalfa wax, paraffin wax, ozokerite, plant waxes, absolute waxes of flowers, animal waxes, marine waxes, polyethylene waxes, and polyolefin waxes.

172. The composition according to claim 171, wherein said plant waxes are chosen from olive tree wax, rice wax, and hydrogenated jojoba wax.

173. The composition according to claim 171, wherein said absolute waxes of flowers are chosen from essential waxes of blackcurrant flower.

174. The composition according to claim 171, wherein said animal waxes are chosen from beeswaxes.

175. The composition according to claim 96, wherein said at least one conditioner is present in an amount ranging from 0.001 % to 20% by weight relative to the total weight of said composition.

176. The composition according to claim 175, wherein said at least one conditioner is present in an amount ranging from 0.01% to 10% by weight relative to the total weight of said composition.

177. A process of caring for and/or treating a keratin material comprising applying to said keratin material a composition comprising:
(1) at least one conditioner chosen from:
synthetic oils;
animal oils;
plant oils;
fluoro oils;
perfluoro oils;
natural waxes; and
synthetic waxes; and
(2) at least one silicone copolymer with a dynamic viscosity ranging from 1×10⁶ to 100×10⁶ cP, resulting from the addition reaction, in the presence of a catalyst, of:
(a) at least one polysiloxane of formula (II): 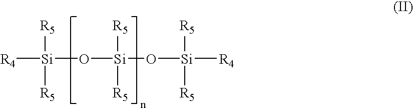
wherein:
R₄, which may be identical or different, are independently chosen from groups that can react by chain addition reaction,
R₅ in formula (II), which may be identical or different, are independently chosen from alkyl, alkenyl, cycloalkyl, aryl, hydroxyl, and alkylaryl groups, and can optionally further comprise at least one functional group,
n is an integer wherein the polysiloxane of formula (II) has a kinematic viscosity ranging from 1 to 1×10⁶ mm²/s; and
(b) at least one silicone compound comprising at least one and not more than two groups capable of reacting with the groups R₄ of the at least one polysiloxane of formula (II), wherein:
at least one of the at least one polysiloxane of formula (II) and the at least one silicone compound (b) comprises an aliphatic group comprising an ethylenic unsaturation;
wherein the composition is in a form chosen from a rinse-out conditioner, a leave-in conditioner, a composition for permanent-waving the hair, a composition for straightening the hair, a composition for dyeing the hair, a composition for bleaching the hair, a rinse-out composition to be applied before a procedure chosen from dyeing, bleaching, permanent-waving and straightening the hair, a rinse-out composition to be applied after a procedure chosen from dyeing, bleaching, permanent-waving and straightening the hair, a rinse-out composition to be applied between the two steps of a permanent-waving operation, and a rinse-out composition to be applied between the two steps of a hair-straightening operation, an aqueous lotion, an aqueous-alcoholic lotion, a gel, a milk, a cream, an emulsion, a thickened lotion, and a mousse.

178. The process according to claim 177, wherein said keratin material is chosen from hair, skin, eyelashes, eyebrows, nails, lips, and scalp.

179. A process for manufacturing a-cosmetic product comprising including in said product:
(1) at least one conditioner chosen from:
synthetic oils;
animal oils;
plant oils;
fluoro oils;
perfluoro oils;
natural waxes; and
synthetic waxes; and
(2) at least one silicone copolymer with a dynamic viscosity ranging from 1×10⁶ to 100×10⁶ cP, resulting from the addition reaction, in the presence of a catalyst, of:
(a) at least one polysiloxane of formula (II): 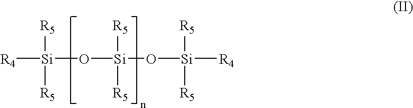
wherein:
R₄ which may be identical or different, are independently chosen from groups that can react by chain addition reaction,
R₅ in formula (II), which may be identical or different, are independently chosen from alkyl, alkenyl, cycloalkyl, aryl, hydroxyl, and alkylaryl groups, and can optionally further comprise at least one functional group,
n is an integer wherein the polysiloxane of formula (II) has a kinematic viscosity ranging from 1 to 1×10⁶ mm²/s; and
(b) at least one silicone compound comprising at least one and not more than two groups capable of reacting with the groups R₄ of the at least one polysiloxane of formula (II), wherein:
at least one of the at least one polysiloxane of formula (II) and the at least one silicone compound (b) comprises an aliphatic group comprising an ethylenic unsaturation;
wherein the composition is in a form chosen from a rinse-out conditioner, a leave-in conditioner, a composition for permanent-waving the hair, a composition for straightening the hair, a composition for dyeing the hair, a composition for bleaching the hair, a rinse-out composition to be applied before a procedure chosen from dyeing, bleaching, permanent-waving and straightening the hair, a rinse-out composition to be applied after a procedure chosen from dyeing, bleaching, permanent-waving and straightening the hair, a rinse-out composition to be applied between the two steps of a permanent-waving operation, and a rinse-out composition to be applied between the two steps of a hair-straightening operation, an aqueous lotion, an aqueous-alcoholic lotion, a gel, a milk, a cream, an emulsion, a thickened lotion, and a mousse.
